(19) United States
(12) Patent Application Publication
(10) Pub. No.: US 2026/0207246 A1
(43) Pub. Date: Jul. 23, 2026

(54) MEDICAL GENERATOR WITH GRID POWER FAILURE DETECTION

(71) Applicant: Erbe Elektromedizin GmbH, Tuebingen (DE)

(72) Inventors: Thomas EGLER, Reutlingen (DE); Thomas RIPPLINGER, Gomaringen (DE)

(21) Appl. No.: 19/448,229

(22) Filed: Jan. 14, 2026

(30) Foreign Application Priority Data
Jan. 17, 2025 (EP) 25152594.5

(51) Int. Cl. A61B 18/12; A61B 18/00

(52) U.S. Cl. CPC A61B 18/1233; A61B 2018/00702; A61B 2560/0209

(57) ABSTRACT

Disclosed herein are generators that are, for example, separated from the power grid during the operation, can be connected with the grid again without concerns or additional precaution measures. For example, apparatus connectors pulled out during operation can be plugged into the apparatus again or power switches actuated during the operation can be switched on again. The control device can then access data and measurements and settings that have been stored during the organized shutdown operation.

### CROSS-REFERENCE TO RELATED APPLICATIONS

This application claims priority to European Patent Application No. 25152594.5, filed Jan. 17, 2025, the entirety of which is incorporated herein.

### BACKGROUND

The invention refers to a medical generator comprising a grid power failure detection.

From EP 2 853 217 B1 a basic configuration of a surgical generator is known. It comprises on an input side a rectifier and a power factor correction circuit supplying a direct voltage link, to which a direct voltage converter is connected. The power factor correction circuit and the direct voltage converter together form a current supply unit for the generator. The power factor correction circuit communicates via a data interface with a system control device to have the ability for carrying out quick power adjustments.

Additional prior art is known from EP 2 475 319 B1.

Besides the power supply unit, medical generators comprise a functional module comprising, for example, a high-frequency oscillator for producing powerful HF currents, with which one or multiple instruments can be supplied for achieving surgical effects on a patient. The control of the functional module and, where applicable, also the current supply unit is provided by a system control device that typically operates in program-controlled manner.

Generators of this type are widespread. In case of unexpected power outage, unintentional power interruption, for example due to pulling out the mains connector, other interruptions of the power supply or also unintentional switch-off during the operation, data can be lost in the system control and undesired conditions can occur.

### BRIEF SUMMARY

Starting therefrom it is the object of the invention to provide a power-failure-proof medical generator.

The object is solved by means of generator embodiments disclosed herein.

The medical generator according to the invention, in one example, comprises a power grid monitoring device that is connected with a grid voltage input on an input side and comprises an evaluation circuit monitoring the presence of the grid voltage. It is configured to produce a shutdown signal in case of the loss of the grid voltage during the operation, wherein the shutdown signal is appropriate to switch off the functional module in order to transfer the latter in a safe condition before the power supply unit is no longer able to supply the functional module with power from the storage and thus the energy stored therein. The shutdown of the functional module and the transfer in a safe condition can be the closing of media valves, where applicable in a predefined sequence, data backup, for example sensor data or data from the progress of a control program running in a control device (for example about the already carried out treatment duration or the like). For example, in a generator for argon plasma coagulation it can be provided that upon receipt of a shutdown signal, immediately the oscillator is switched off that supplies the instrument with power, whereas the valve for supplying the instrument with argon is closed only subsequently. Diverse other shutdown sequences are possible.

The storage can comprise one or multiple storage capacitors (for example electrolytic capacitors), which are arranged at one or multiple positions of the circuit of the generator, particularly in the power supply unit. For example, the power supply unit can comprise a power factor correction circuit that supplies a direct voltage link to which one or more capacitors are connected. To the direct voltage link one or more direct voltage converters can be connected, which can also be provided with capacitors or capacitor groups on the output side. The term “storage” thereby relates to those capacitors or capacitor groups from which a unit that has to be transferred into a safe condition-is to be supplied in case of power failure. This unit can be an entire functional module or also only a part thereof, for example the control device, which can be part of the functional module.

The power supply unit is able, due to its storage, to supply the functional module via a supply period of, for example, 100 milliseconds (ms) or also longer with operating current if the grid voltage input is currentless. Due to a respective dimensioning of the storage, the supply period is at least so long that the functional module and particularly its control can be transferred into a safe shutdown condition within the supply period. Running programs can be terminated in organized manner and data can be saved, for example sensor data, operating data or the like.

Preferably, the power grid monitoring device is configured to produce the shutdown signal only after a preset waiting period, wherein the waiting period is shorter than the supply period. Thereby the difference between the supply period and the waiting period is longer than the shutdown period required for the transfer of the functional module or its control.

Due to the setting of the waiting period, it can be avoided that the power grid monitoring device creates a shut down signal based on irrelevant grid interferences. The generator is thus tolerant against minor grid disturbances, which are per se innocuous. Such irrelevant and thus tolerable grid disturbances can be, for example, the complete or partial loss of power half-waves or other power grid disturbances, for example short-term voltage fluctuations. If the waiting period has an amount of, for example, 10 or a little more than 10 milliseconds, the loss of the entire power half-wave does not yet result in production of a shutdown signal. However, if the grid voltage fails completely, a shutdown signal is produced after termination of the waiting period and the control of the functional module is transferred into a safe condition. The grid voltage loss can be a general blackout. It can also be caused by unintentionally pulling out the mains connector of the generator (for example due to moving the generator inside the operation room) or due to unintentional actuation of the main power switch of the generator.

The control device provided for control of the functional module and the entire generator can comprise one (or more) controller(s) (computational units), which is or are configured for processing a control program and operate controlled by the control program. The control program is configured to control the supply of voltage, current or also media, such as gas or liquids, to connected instruments and to thereby realize the desired operation mode. Operation modes determine the surgical effect that is to be achieved on the patient. For each mode specific electrical characteristics of the power supply to the instrument are predefined and can only be varied in predefined limits. Such electrical characteristics are, for example, the voltage, the frequency, the modulation type, the pulse-pause ratio in case of on-off-modulation, limits for the current strength, power limits, temporal progresses of the indicated parameters and the like.

The control program is configured to initiate and process a shutdown sequence upon receipt of a shutdown signal. In the context of the shutdown sequence the operation of the functional module can be terminated, gained sensor data and, where applicable, other data can be backed-up and running programs can be terminated if necessary. Due to storing the data, the control program can continue its operation in organized manner if the grid voltage returns. For example, the control program can be configured to store user inputs and/or program data, for example about the set mode, upon receipt of the shutdown signal in the context of the shutdown sequence. In doing so, the set mode is maintained over the power grid voltage failure. Treatment data recorded in the mode, for example the application duration or the application intensity, are also maintained, so that the operation of the functional module can be continued in safe manner for the patient after return of the power grid voltage.

The shutdown sequence can also provide that immediately after receipt of the shutdown signal, powerful loads, such as the oscillator for supply of surgical instruments, are switched off so that the energy present in the storage can be predominantly used for further supplying the system control or another control device. Thereby the shutdown of the control device (particularly the entire system control) and saving of all necessary data is simplified without the need to provide separate energy storages for this purpose.

The power grid monitoring device is preferably connected with the grid voltage input via a full-wave rectifier, so that the presence of both half-waves of the grid voltage is monitored. Between the grid connection and the full-wave rectifier a net filter can be arranged having one or more reactive components, for example inductors that couple or do not couple with each other and particularly capacitors, in order to avoid the propagation of electrical disturbances from the generator into the grid and from the grid into the generator.

The power grid monitoring device is configured to detect a low ohmic as well as a high ohmic grid voltage failure. It is particularly configured to reliably detect these different failure scenarios even if in case of a high ohmic voltage failure residual voltages are present in the grid filter due to the energy storing components that are present there.

A low ohmic grid voltage failure is present if the grid voltage breaks down to zero, so that the two grid lines practically are on equal potential (“blackout”) or it is reduced so far that a correct operation of the generator is no longer possible (“brownout”) . A high ohmic grid voltage failure is present if, for example, the mains connector is unintentionally pulled out of the socket or if the apparatus connector is pulled out of the generator. Thereby direct voltage components can be applied at least temporally to the grid voltage input of the power supply unit and thus to the input of the power grid monitoring device, wherein the direct voltage components can come from charged capacitors of the grid filter on the input side, for example.

Both failure scenarios are detected by the power grid monitoring device. For this purpose, the latter comprises a high pass connected to the rectifier on the input side, which does not allow direct voltage to pass. In addition, the power grid monitoring device can comprise impulse forming circuits as well as a clock generator circuit that determines the waiting period. The clock generator circuit can be configured as monoflop that can be retriggered, which creates the shutdown signal if it is not triggered within the waiting period from the grid side by a new impulse. These impulses are newly produced by each power half-wave.

Further details of advantageous embodiments of the invention are subject matter of the claims as well as the description and the accompanying drawings.

### BRIEF DESCRIPTION OF THE SEVERAL VIEWS OF THE DRAWING(S)

FIG. 1 a medical generator according to the invention provided with power grid failure detection in a schematic illustration,

FIG. 2 the power supply unit of the generator according to FIG. 1 in a basic circuit diagram,

FIG. 3 the power supply unit in schematic and simplified illustration as well as the assigned power grid monitoring device,

FIG. 4 the temporal progress of the rectified grid alternating voltage in an exemplary diagram and

FIG. 5 the temporal progress of the supply voltage and the shutdown signal in accordance with the grid voltage according to FIG. 4 in form of a diagram.

### DETAILED DESCRIPTION

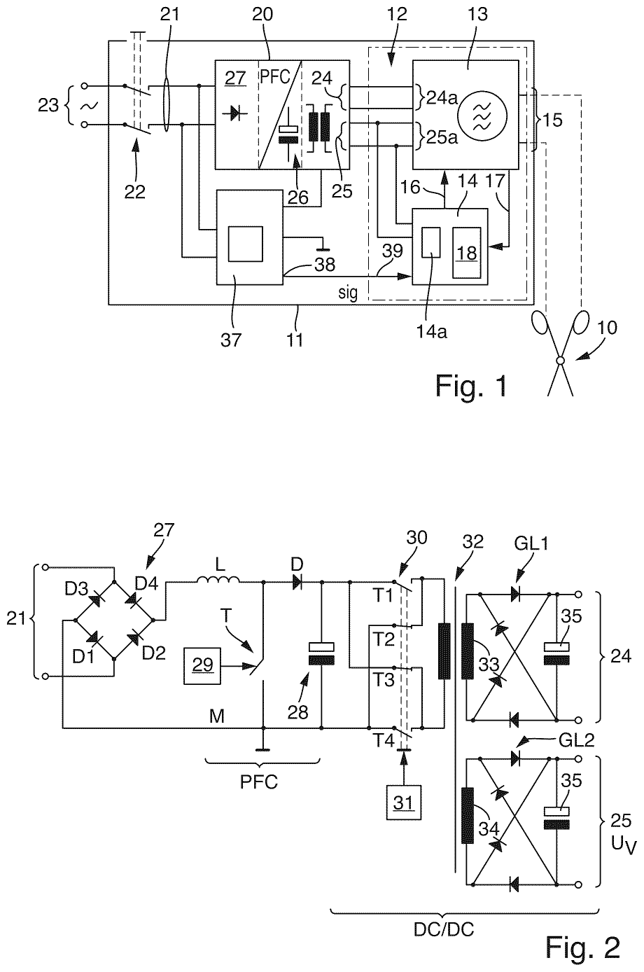

In FIG. 1 an instrument 10 for treating a human or animal patient as well as a generator 11 are schematically illustrated, the latter serving for supply of the instrument 10 with power. The instrument 10 can be an electrosurgical instrument configured in monopolar or bipolar or multipolar manner. For example, it can serve for coagulating, cutting, fusioning of tissue or for carrying out another electrosurgical surgery. The generator 11 can, apart from the supply with power for electrosurgical operations, also be configured for supply of the instrument 10 with other media, for example liquids, vapors, aerosols or gases, for example argon. The generator can also comprise a suction device, for example in order to suck gases, fume or liquids from a field of operation. The instrument 10 can be an instrument for argon plasma coagulation or also a cryosurgical instrument or the like.

For supply of the instrument 10 generator 11 comprises a functional module 12 that provides one or more of the indicated media (power, gas, liquid) for instrument 10.

In FIG. 1 functional module 12 is exemplarily illustrated only as a purely electrical supply module having a high frequency oscillator 13 and a control device 14. The control device can also be configured to control the entire generator and thus form a system control. The functional module 12 can, however, additionally or alternatively also comprise additional or other supply modules, for example a source for cryofluids, flushing fluids or others.

The oscillator 13 comprises at least one output 15 to which the instrument 10 is connected in order to be supplied with high voltage for carrying out the desired surgical purpose and a respective current. This preferably involves high frequency alternating current having a frequency of above 200 kHz, however, at least preferably, below 5 MHz. For producing the HF voltage the HF oscillator 13 can have any known circuit configuration.

The HF oscillator 13 (as well as also any other supply module) operates under the control of control device 14, which is configured to set characteristics of the current or the voltage created by the HF oscillator 13. Such characteristics can be, for example, the height of the voltage and/or the current, the output power, the type of the modulation and the modulation depth impressed on the current, the crest factor and others. In doing so, control device 14 can set the operating mode, for example coagulation, cutting, tissue fusion or the like. In addition, in or on HF oscillator 13 sensor units can be provided for obtaining data, for example about the amount of the voltage output to the instrument, of the current, the treatment duration, of the power, the tissue resistance and the like. These sensor units can forward respective data to the control device 14. The control of oscillator 13 by control device 14 is symbolized by means of an arrow 16. The transmission of data from the oscillator 13 to the control device 14 is symbolized by means of an arrow 17.

For control of HF oscillator 13 the control device 14 comprises a programmable unit, such as a controller 14a, a computing unit or a network of controllers or computers. The controller or controllers 14a or computer(s) are configured to operate in program-controlled manner. In addition, the control device 14 comprises one or more not illustrated data memories for recording and storing one or more programs, data coming from processing the programs as well as for storing data supplied from the sensor units (arrow 17).

The control device 14 comprises in addition an input and output device 18, comprising operating elements for user input and output elements. The input elements can be keys, buttons, touchscreens or the like. The output means can be displays for representing optical information, control lamps, measuring instruments or the like. The input and output means can however also be arranged external from generator 10 and can be formed by a portable apparatus that is connected with generator 10 by means of a wired or wireless data connection.

The functional module 12 is connected to a power supply unit 20, the grid voltage input 21 of which is connected with the general electrical supply grid 23 at least via an optionally provided power switch 22. The power switch 22 is configured to selectively close or interrupt at least one, however preferably both of the lines leading to the grid voltage input. Between the power switch 22 and the grid voltage input 21 typically a grid filter is provided. For connection between generator 10 and supply grid 23 a connection cable, for example a commercially available cold-device cable having a grid side connector and an apparatus side connector.

The alternating voltage grid is typically a grid having a grid frequency of 50 Hz or 60 Hz and an effective voltage between 110 Volts and 240 Volts. Basically, the invention is however suitable also for use in generators for other grids.

The power supply unit 20 comprises at least one, typically however multiple operating voltage outputs 24, 25 in order to supply the functional module 12 at respective operating voltage inputs 24a, 25a with the required operating voltages. The operating voltages are direct voltages. For example, a direct voltage of multiple hundred volts can be applied to the operating voltage output 24, whereas at the operating voltage output 25 only a direct voltage, for example 3, 5, 12 or 24 V, applies that is suitable for operation of controls including the control device 14,. Other voltages are possible depending on the configuration of the respective controls.

The power supply unit 20 is connected with the supply grid 24 with its grid voltage input 21 via power switch 22 in order to draw the required operating power therefrom.

The voltage applying at the operating voltage outputs 24 and 25 is preferably a stabilized direct voltage—the HF oscillator 13 as well as the control device 14 are supplied with direct current.

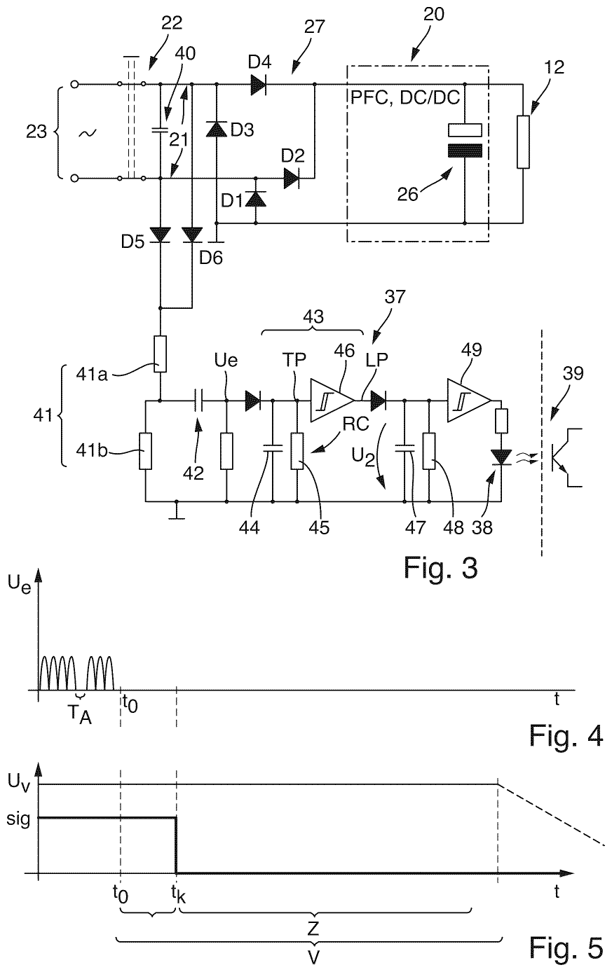

The power supply unit 20 comprises a storage 26 from which the functional module 12, symbolized as ohmic load in FIG. 3, can be supplied with operating power during a supply period V (FIG. 5) if the power supply unit 20 does not receive current from the supply grid 23 at its grid voltage input 21. For example, such a situation can be an unexpected failure of the grid voltage, an unintentional or also intentional unplugging of the grid connector of generator 11 or the actuation of the power switch 22. The supply period V thereby results from the capacity of storage 26, the load stored therein as well as the maximum power requirement of functional module 12 during the processing of a shutdown sequence.

For the invention it is important that at least the entirely or partly program-controlled control device 14 is supplied with operating voltage (and current) during the supply period V (during the shutdown sequence) in case of loss of the grid voltage. On the contrary, for the HF oscillator 13 it can be accepted if the operating voltage provided for the latter drops down within the supply period Z. However, in order to not create undesired surgical effects and thus damages on the patient in the context of the shutdown sequence it can be provided that also the voltage applied at the operating voltage output 24 for operation of the RF oscillator 13 is maintained during the supply period Z. Alternatively it can be provided that the HF oscillator is turned off immediately after receipt of the shutdown signal in the context of the shutdown sequence, in order to consume as little energy as possible during the shutdown sequence.

The storage device 26 may consist of one or more capacitors, preferably electrolytic capacitors, which may be located at different positions in the power supply unit. Reference is made to FIG. 2, which shows a schematic diagram of the basic structure of power supply unit 20.

The power supply unit 20 comprises a full-wave rectifier 27 on its input side, for example in form of a full-wave bridge (Graetz bridge) adjoined by a power factor correction circuit (PFC). Part of the latter are an inductor L and a diode D that are connected in series with each other and that preferably connect the positive output of the full-wave rectifier 27 with the positive connection of a storage capacitor 28. The connection point between the inductor L and the diode D is connected to an electronic switch T—for example in form of a bipolar transistor, a field effect transistor or an insulated-gate bipolar transistor (IGBT)—with its emitter or source connected to ground M just like the negative connection of the storage capacitor 28. The electronic switch T comprises a control electrode connected to a PFC clock generator 29. By periodically opening and closing of the electronical switch T the storage capacitor 28 is charged to a direct voltage that is higher than the peak voltage of the grid alternating voltage.

A direct voltage converter DC/DC, which is connected with storage capacitor 28 on the input side, adjoins the power factor correction circuit PFC, wherein the direct voltage converter DC/DC also serves for galvanically isolating the operating voltage outputs 24, 25 from the grid voltage input 21. In principle any direct voltage converter of known configuration can be used for this purpose. In the present embodiment the direct voltage converter DC/DC comprises a full-wave inverter 30 having four controlled electronic switches T1, T2, T3, T4 in total, which are controlled by an inverter clock generator 31. Connected to the full-wave inverter 30 is the primary winding of a transformer 32, which can comprise one or more secondary windings 33, 34. The latter supply storage capacitors 35, 36 connected to the respective operating voltage outputs 24, 25 via full-wave rectifiers GL1, GL2.

For the further description of the illustrated embodiment, first it is assumed that—in case of a sudden grid failure—it is important for the organized shutdown of generator 11 and thus also for the organized restart of the generator that at least the control device 14 is supplied so long with operating power until the program running on the control device 14 has been terminated in an organized manner and the data are backed up, for example data received from the HF oscillator 13 as well as other data. In this case the relevant storage 26 is formed by storage capacitor 36 and storage capacitor 28. The latter is in terms of its capacity converted based on the transmission factor between the primary winding of the transformer 32 and the secondary winding 34 minus possible efficiency losses. In the event of grid failure the storage 26 is suitable to supply the control device 14 over the supply period V and thus so long or longer with power, as it is necessary for the organized termination of the operation of control device 14 and the transfer into a defined shutdown condition.

According to the invention generator 11 comprises a power grid monitoring device 37 that is configured to provide a shutdown signal sig to an input 39 of functional module 12 from an output 38 if loss of the grid voltage at the grid voltage input 21 has been determined. The functional module 12 is thereby configured to initiate the shutdown sequence particularly of control device 14 upon receipt of the shut down signal sig, wherein the control device 14 then backups data and terminates running operating programs. Part of this can also be the shutdown of HF oscillator 13 and, where applicable, the output of optical and/or acoustical and/or other signals that signal a treating person that generator 10 is no longer operative. It can also be signaled that a possibly started surgical measure has not yet been completed correctly, for example a coagulation process or a fusion process, for example during sealing of vessels.

The power grid monitoring device 37 is apparent from FIG. 3. The power supply unit 20 formed by power factor correction circuit PFC and direct voltage converter DC/DC is only schematically illustrated together with its storage 26. On the input side in turn the full-wave rectifier (Graetz bridge) consisting of diodes D1 to D4 is illustrated preceded by a net filter only illustrated by a capacitor 40.

In addition, diodes D5, D6 are connected to the alternating voltage input 21 that form a second Graetz bridge, that means a full-wave rectifier, together with diodes D1 and D3 of first Graetz bridge (full-wave rectifier 27). The rectified voltage drawn from grid voltage input 21 is supplied to an impulse forming circuit 43 via a voltage divider 41, formed by two or more resistors 41a, 41b and serving for level adaption, and a capacitor 42 serving for filtering out direct voltage components. A capacitor 44 and a discharge resistor 45 connected in parallel thereto, thereby forming an RC element, are part of the impulse forming circuit 43. A trigger circuit 46 is connected to the RC element that together with the RC element forms the impulse former 43.

The impulse former 43 is connected on its output side to another impulse former having another RC element consisting of a capacitor 47 and a discharge resistor 48, to which again a trigger circuit 49 is connected. The latter is connected on the output side with the light emitting diode of an optocoupler forming the output 38 of the power grid monitoring device 37.

The generator 11 described so far and particularly the power grid monitoring device 37 operate as follows:

Case 1: The grid voltage of supply grid 23 breaks down completely during the operation of generator 11. Thus, the voltage at the alternating voltage input 21 drops down to zero practically immediately. The power factor correction circuit PFC ceases to operate and as a consequence the storage capacitor 28 does not receive any additional charge. However, the direct voltage converter DC/DC can continue to operate at least as long as the voltage provided on the storage capacitor 28 is sufficient for its operation. In doing so, the direct voltage converter DC/DC can at least transfer part of the charge of storage capacitor 28 to capacitor 36.

It is assumed that the breakdown of the grid voltage happens at the point in time to marked in FIG. 5. Prior to this, that means prior to the loss of the grid voltage, each power half-wave had provided charge on capacitor 44 via one of the diodes D5 and D6, wherein the capacitor 44 has been practically immediately discharged again by discharge resistor 45. Accordingly at the output of trigger circuit 46 power half-wave synchronous charging impulses have been available in order to charge the RC element formed by capacitor 47 and resistor 48. As long as it maintained charged, trigger circuit 49 signaled the presence of the grid voltage at the output 38, for example in that the light emitting diode of the optocoupler arranged there was illuminated. Insofar the power grid monitoring device can be considered as retriggerable monoflop that has been continuously newly triggered by the impulses provided at the output of the trigger circuit 46 derived from the power half-waves. If these impulses however get lost starting with the point in time to a certain discharge time for the capacitor 47 goes by after the termination of which the trigger circuit 49 switches (that means diode 38 extinguishes). The extinction of diode 38 is the signal sig for switching off functional module 12.

The period between the trigger impulses at the point in time to and the switching of the trigger circuit 49 and thus the extinction of the light emitting diode at the output 38 is a waiting period, which is denoted in FIG. 5 by tₖ. In other words, after a grid voltage loss the waiting period K is waited for and the shutdown signal sig is created at the point in time tₖ.

The definition of the waiting period has the advantage that very short-term grid disturbances, as they are present in the period T_{A} in FIG. 4, do not result in a shutdown process. An interfering impulse or also the loss of an entire power half-wave does not result in the creation of the shutdown signal sig. However, if subsequently multiple power half-waves get lost in a time interval, the total duration of which is longer than the waiting period K, the signal sig is created.

The functional module 12 receives the shutdown signal sig and initiates the shutdown sequence. Particularly, the control device 14 backs up present data and program conditions in the context of the shutdown sequence in order to make an organized continued operation possible after restart.

From the dimension of storage 26 and the power requirement of functional module 12 or at least the control device 14 the supply period V results during which the operation of control device 14 can be guaranteed by means of the energy stored in storage 26. The shutdown period Z required for the organized shutdown, particularly of control device 14, is preferably shorter than (or at most as long as) the difference between the supply period V and the waiting period K. This is guaranteed due to a respective dimensioning of the storage capacitors 28 and 36 (FIG. 2). In this regard FIG. 5 illustrates that the supply voltage UV starts to decrease only after termination of the supply period V.

Case 2: An intentional or unintentional unplugging of the power connector and thus the separation of generator 11 from the supply grid 23 or also the opening of power switch 22 also results in a loss of the supply power otherwise provided by the grid. Thus, also in this case via diodes D5, D6 voltage half-waves are no longer transmitted to the power grid monitoring device 37. The capacitor 42 thereby also avoids that a direct voltage that is possibly provided on capacitor 40 (that means in the net filter) simulates the presence of the grid voltage. For this reason, the proceedings in this scenario insofar entirely correspond to the scenario described above.

The medical generator according to the invention comprises a power supply unit 20 having a storage capability, wherein the storage capability is sufficient to allow at least the control device 14 for a period V during which data, measurements, program conditions and settings or the like present in the control device 14 can be backed up and can be stored for an organized continued operation. For determination of a grid voltage breakdown or also a separation of the generator 11 from the supply grid 23 a power grid monitoring device 37 is provided that continuously monitors the grid voltage and outputs a shutdown signal sig as soon as the grid voltage is no longer present for a time period that is longer than a waiting period. The waiting period is preferably longer than the duration of a power half-wave, preferably longer than the duration of a complete power wave.

With the concept according to the invention generators 11 that are, for example, separated from the power grid during the operation, can be connected with the grid again without concerns or additional precaution measures. For example, apparatus connectors pulled out during operation can be plugged into the apparatus again or power switches actuated during the operation can be switched on again. The control device 14 can then access data and measurements and settings that have been stored during the organized shutdown operation.

1. A medical generator comprising:
a power supply unit, which comprises a grid voltage input connectable to an alternating voltage power supply grid and which comprises at least one operating voltage output ;
at least one functional module, which comprises at least one operating voltage input, connected to the at least one operating voltage output and which comprises at least one instrument output adapted to be connected to a surgical instrument,
wherein the power supply unit comprises a storage that is configured to supply the functional module over a predetermined supply period (V) with operating current in case of a currentless grid voltage input; and,
a power grid monitoring device, which comprises an input connected with the grid voltage input and which is configured to produce a shutdown signal (sig) and to transmit it to the functional module in order to transfer the functional module within a shutdown period (Z) into a safe condition in case of loss of the grid voltage.

2. The medical generator according to claim 1, wherein the power grid monitoring device is configured to produce the shutdown signal (sig) only after a predefined waiting period (K), wherein the waiting period (K) is shorter than the supply period (V).

3. The medical generator according to claim 2, wherein a difference between the supply period (V) and the waiting period (K) is longer than the shutdown period (Z) required by the functional module for the transfer into the safe condition.

4. The medical generator according to claim 1, wherein the functional module comprises at least one controller that operates in a manner controlled by a control program.

5. The medical generator according to claim 4, wherein the control program is configured to receive the shutdown signal (sig) and to terminate an operation of the functional module in response to the shutdown signal (sig).

6. The medical generator according to claim 2, wherein the power grid monitoring device is connected with the grid voltage input via a full-wave rectifier (D1, D3, D5, D6) and is; on an output side; connected with a clock generator circuit.

7. The medical generator according to claim 6, wherein the clock generator circuit is configured in a manner that determines the waiting period (K).

8. The medical generator according to claim 6, wherein the clock generator circuit is configured as re-triggerable monoflop.

9. The medical generator according to claim 6, wherein the power grid monitoring device comprises at least one grid triggered impulse forming device that is configured to create a charge impulse (LP) for each power half-wave generating a trigger impulse (TP), wherein the charge impulse (LP) is forwarded to an RC element having a storage capacitor and a discharge resistor connected in parallel thereto, wherein the RC element defines a time constant (τ) and wherein the storage capacitor is charged by each charge impulse on a predetermined charge voltage (U_{L}).

10. The medical generator according to claim 9, wherein downstream of the RC element a threshold switch is provided having a predefined switching threshold.

11. The medical generator according to claim 10, wherein the charge voltage (UL), the switching threshold and the time constant (τ) are coordinated with each other, so that the switching threshold is achieved only after cessation of multiple charging impulses (LP).

12. The medical generator according to claim 1, wherein the power supply unit comprises a grid rectifier that is connected on an input side to the grid voltage input and comprises an output to which a power factor correction circuit (PFC) is connected.

13. The medical generator according to claim 12, wherein the power factor correction circuit (PFC) is connected in supplying manner with at least one storage capacitor, which is part of the storage.

14. The medical generator according to claim 13, wherein a direct voltage converter (DC/DC) is connected to the storage capacitor and comprises on an output side a storage capacitor that is part of the storage.

15. The medical generator according to claim 14, wherein the direct voltage converter (DC/DC) is configured in potential isolating manner.
